(19) United States
(12) Patent Application Publication
(10) Pub. No.: US 2025/0101072 A1
(43) Pub. Date: Mar. 27, 2025

(54) METHOD OF USING/APPLYING A KERATIN HYDROLYSIS PEPTIDE SOLUTION TO ENAHNCE THE FLAVOR OF TEA LEAVES

(71) Applicant: CH Biotech R&D Co., Ltd., Nantou City (TW)

(72) Inventors: Iou-Zen CHEN, Taipei (TW); Meng-Ying LI, Nantou (TW); Pei-Chun LIAO, New Taipei (TW); Nai-Hua YE, Chunghua (TW); Ming-Yuan LEE, Nantou (TW)

(73) Assignee: CH Biotech R&D Co., Ltd., Nantou City (TW)

(21) Appl. No.: 18/533,181

(22) Filed: Dec. 8, 2023

(30) Foreign Application Priority Data
Sep. 22, 2023 (TW) 112136374

(51) Int. Cl. C07K 14/47; A01H 1/00; A23F 3/40; C07K 1/12; G01N 33/68

(52) U.S. Cl. CPC C07K 14/4741; A01H 1/10; A01H 1/1225; A23F 3/405; C07K 1/12; G01N 33/6848; G01N 2333/4742

(57) ABSTRACT

Present invention teaches the method of using a keratin hydrolysis peptide (“KHP”) solution to enhance sweetness and flavors of tea leaves. By selectively choosing specific weights of feathers and water, and treating the mixture to a high-temperature high-pressure hydrolysis process, the resulting solution is confirmed to contain at least 253 peptides and then applied to the surface of tea leaves during sprouting stage and infused to the soil around the tea trees/plants; the increased content of L-theanine and polyphenol is separately tested and confirmed. Optionally, the KHP solution can be diluted by water, as taught in the specification, before applying to the tea leaves and the soil as taught herein.

### PRIORITY CLAIM TO FOREIGN APPLICATION

Applicant hereby makes priority claim to a Taiwan application, number 112134374, having the Taiwan filing date of Sep. 22, 2023.

### BACKGROUND OF THE INVENTION

Present invention disclosed and claimed the method and application of a keratin hydrolysis peptide (“KHP”) solution to tea trees/plants, resulting in the enhancement of the flavor that's usually the tasting senses brought by the L-theanine and polyphenol content.

The KHP solution is made by a hydrolysis process using feathers and water, with one version of the solution using feathers only, via a high-temperature and high-pressure process, resulting in a solution that has many beneficial applications in the fields of horticulture, agriculture and potentially other farming businesses.

Hydrolyzed keratin has long been used to strengthen hairs, reduce breakage, and minimize damages. Some form of chemical process is used to break down the large protein molecule of the hydrolyzed keratin so that it can penetrate the hair cuticles and do its magic.

Hydrolyzed keratin solution has also been used to help to boost the production of certain plants and crops. The inventors of present application found, by research and experiment, that certain method of keratin hydrolysis and the application may lead to improving tea leaves' taste.

Tea drinking history dated back hundreds of year, in many places of the world. Usually, the harvested tea leaves go through different dried/baked process, then are steeped in hot water, or cooked in hot water, with optional milk, sugar or other ingredients added, before serving/drinking. Not only are the flavor and the sweetness enjoyed by people, it is also a healthy drink.

The popularity of the tea drinking history also caused the growing and harvesting knowhow to develop over many generations. Through modern technology, people learned that it is the L-theanine and polyphenol content in the tea leaves that are directly tied to the flavor, aroma and sweetness enjoyed by tea drinkers. The polyphenol is a group of fragrant molecular substances with antioxidant attribute. As an ingredient in tea leaves, its adds to the aroma of the tea drinks. As such, there are many competing methods to help with the growth of tea plants that will produce tea leaves with higher L-theanine, polyphenol, free amino acids and other ingredients that would contribute to the enhanced flavors of tea leaves.

Tea plants/trees grow faster in a lower elevation; lower elevation is generally known in the tea farming as under 1,000-meter elevation. As such, growing tea plants are likely to have better yield by planting them in lower elevation. However, while more sunny days are conducive the faster growth, over exposure to sun also causes tea plants to produce more catechins and tea cymbals that tend to give a bitter taste to the tea leaves.

It has been a desired result in the industry to find good solutions so as to reach a good balance of low-elevation tea farming, thus better for the environment, and to obtain the tea leaves with less bitter taste.

In light of the problems faced by the tea farming industry, the inventors of present application experimented and worked on the method of employing a high-temperature and high-pressure hydrolysis process to break down the keratin in feathers, and to form a hydrolyzed peptide solution that can be applied to tea plants, suitable for low-elevation planting, resulting in the tea leaves having better flavor and aroma.

### SUMMARY OF THE INVENTION

While growing tea plants at higher elevation will tend to produce tea leaves with sweeter taste and aroma (higher L-theanine and polyphenol content), it in fact would be a more expensive approach, given the land acquisition cost and overall transportation considerations involving materials, production deliveries, and human labors, etc.

Instead of resorting to chemicals, fertilizers or other traditional method of growing/farming, the inventors turned to the technical solution of making and applying the KHP solution to help with the enhancement of tea leaves in an environmentally friendly manner.

The keratin solution is primarily based upon feather, which contains 85-91% keratin, 13-15% organic nitrogen, 1.6-2% organic sulfur, as well as other materials. The high keratin content has drawn many prior researches that work to break down, by enzyme, chemical agents, or fermentation process, into peptides, amino acids and other smaller molecules that can be used for animal feeds, plant fertilizers, and cultivation bases.

Around 2019, Nurdiawati, et al, came up with a hydrolysis process, by the mixture of a-amylase and protease to hydrolyze feather waste, resulting in a mixture of amino acids, fatty acids, and sugars. Nurdiawati experimented and adopted certain specific high-temperature and high-pressure setting in the hydrolysis process and discovered that the resulting solution, when mixed with some potassium and other minerals, can boost the growth of Pogostemon cablin and Vigna radiata, as reported in International Journal of Recycling or Organic Waste in Agriculture (8:221-232, 2019).

The inventors of present application selected different feather and water compositions and performed the hydrolysis at higher temperature and higher pressure setting, and obtained a different keratin hydrolysis peptide (“KHP”) solution that can be used on different crops/plants.

The inventors further experimented the method of applying the KHP solution, some with further dilution, on the tea plants in the field, as well as other plants and proved the effectiveness of the method in producing tea leaves with better flavors and aromas.

The first embodiment of present invention uses a mixture of water and feathers (water content 50%) at the weight ratio of 5:4 (specific weight of 50 kg water and 40 kg feathers) and subject the mixture to a thermal hydrolysis process (80 minutes) to create a solution, resulting in the solution having molecular mass of 593.3-3,508.9 Dalton and containing at least 253 peptides as confirmed by using a mass spectrometer.

The inventors used the spectrometer Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The solution then is applied to tea plants at leaf sprouting stage.

The solution can be diluted by water and then sprayed to the tea leaves' surface; the solution can also be diluted and infused to the soil around the tea plants.

with some dilution and with optional mixing of nitrogen-based fertilizer, to the cotton plants.

A second embodiment uses mixes 66 kg of feathers (water content 50%) with 44 kg of water, then treat it to a thermal hydrolysis process (40 minutes), resulting in a solution with molecular mass of 593.3-3,828.0 Dalton, containing at least 253 peptides.

The confirmation of at least some of the bioactive peptides is made by further referencing the BIOPEP-UWM database.

The same method of application to the tea plants can also be used on the second embodiment.

### BRIEF DESCRIPTION OF DRAWINGS

The accompanying drawings, figures and tables, which are incorporated in and constitute a part of this specification, illustrate and exemplify the preferred embodiments of the invention. Together with the description, serve to explain the principles of the invention.

Table I shows the at least 253 peptides and its annotated sequences for the solution generated in accordance with the disclosure of this application.

Table II shows the parameters of the hydrolysis for the two embodiments

FIG. 1 shows the comparison of L-theanine content among the three groups, where the tea plants' leaves are sprayed with KHP solutions.

FIG. 2 shows the comparison of polyphenol content, where the tea plants' leaves are sprayed with KHP solution.

FIG. 3 shows the comparison of L-theanine content among the three groups, where the soils around tea plants are infused with KHP solutions.

FIG. 4 shows the comparison of polyphenol content, where the soils around tea plants are infused with KHP solutions.

### DETAILED DESCRIPTION OF THE INVENTION

The keratin hydrolysis peptide (“KHP”) solution of present invention, as a first specific embodiment, is made by a high-temperature and high-pressure process to treat a mixture of water and feathers where the weight ratio whereby the weight ratio of feathers and water is set at 5:4. In the disclosure below, the specific weight of 50 kg feathers and 40 kg water will be used.

The hydrolysis process takes the steps of:
a. Preparing the KHP solution by mixing 50 kg of feathers whose content is 50% water with 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm;
The confirmation of some of the bioactive 253 peptides is further done by referencing the BIOPEP-UWM database.

The solution can then be applied to the tea plants at leaf sprouting stage.

The method of using a keratin hydrolysis peptide (KHP) solution stated above further causes the solution to be diluted with water by volume at the ratio of 1:200-1,000 and is sprayed to the tea leaves' surface.

The method of using a keratin hydrolysis peptide (KHP) solution stated above further causes the solution to be diluted with water by volume at the ratio of 1:100-1,000 and is infused to the soil around the tea plants.

A method of using the solution of present invention, as a second specific embodiment, is made by a high-temperature and high-pressure process to treat a mixture of water and feathers where the weight ratio whereby the weight ratio of feathers and water is set at 6:4. In the disclosure below, the specific weight of 66 kg feathers and 44 kg water will be used.

The hydrolysis process takes the steps of:
a. Preparing the KHP solution by putting 66 kg of feathers whose content is 50% with 40 kg of water in a sealed container water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 3.0×10⁵˜4.5×10⁵ ppm;

The KHP solution stated above is applied to tea plants at leaf sprouting stage.

The method of using a keratin hydrolysis peptide (KHP) solution stated above further causes the solution to be diluted with water by volume at the ratio of 1:200-1,000 and is sprayed to the tea leaves' surface.

The method of using a keratin hydrolysis peptide (KHP) solution stated above further causes the solution to be diluted with water by volume at the ratio of 1:100-1,000 and is infused to the soil around the tea plants.

The inventors of present application conducted actual field tests on the tea plants at different growth stages, by spraying the KHP solutions at the tea leaves or by infusing the soils around the tea plants with KHP solutions.

The tea plants in the field tests are made up of three (3) groups: CK, KHP-1 and KHP-2. Check group (CHK) tea plants are fed with water and grown under normal circumstance, while the KHP-1 and KHP-2, in addition to the normal growing conditions, are given the first and second embodiments solution, and applied as noted herein.

The test location was set in Pingtung County, Taiwan, at 100-meter elevation, with the KHP solutions (both embodiments) diluted with water (by volume) at 1:500 ratio.

Tea leaf samples that are sprayed with KHP-1 and KHP-2 solutions are harvested, at 78 cm height of the plant (first time picked) and at 68 cm height of the plant (second time picked) 20 days after second spraying of the KHP solution, and were then sent for analysis of the amount of L-theanine and polyphenol content.

The harvested tea leaf samples are dipped into pure water, extracted by a centrifuge machine for 30 minutes, at a temperature of 70° C. After adding the same volume of sulfo-water salami solution of 3% strength, the mixed liquid is then analyzed by an amino acid analysis instrument (L-8900, Hitachi) to analyze/measure the L-theanine content.

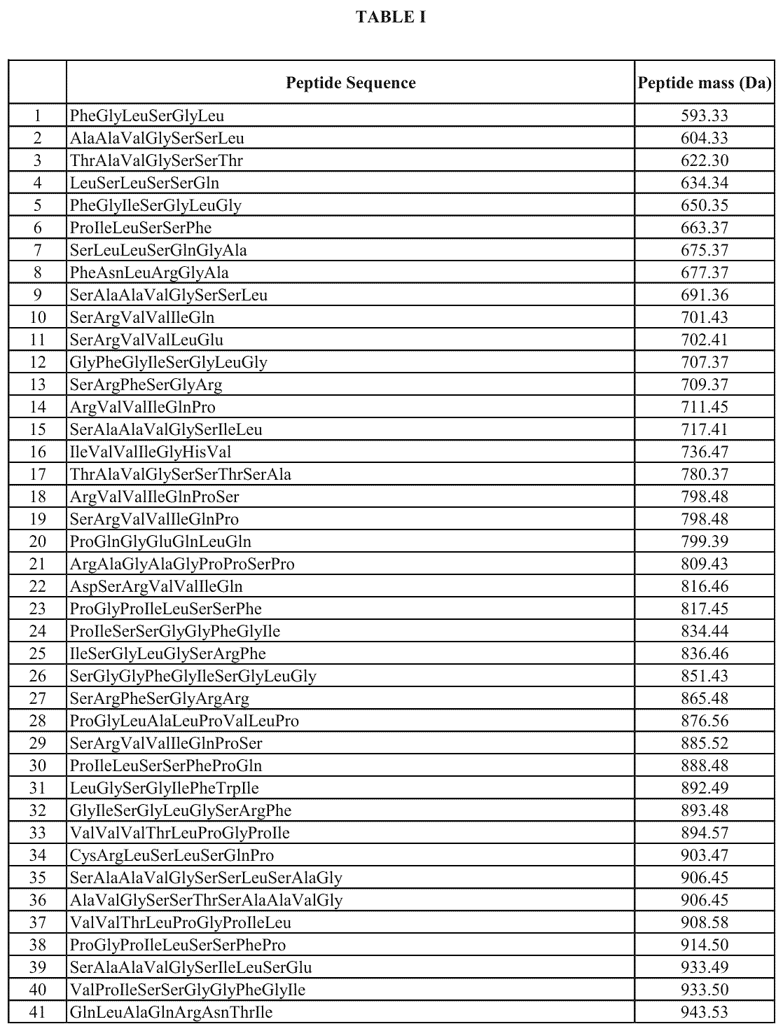

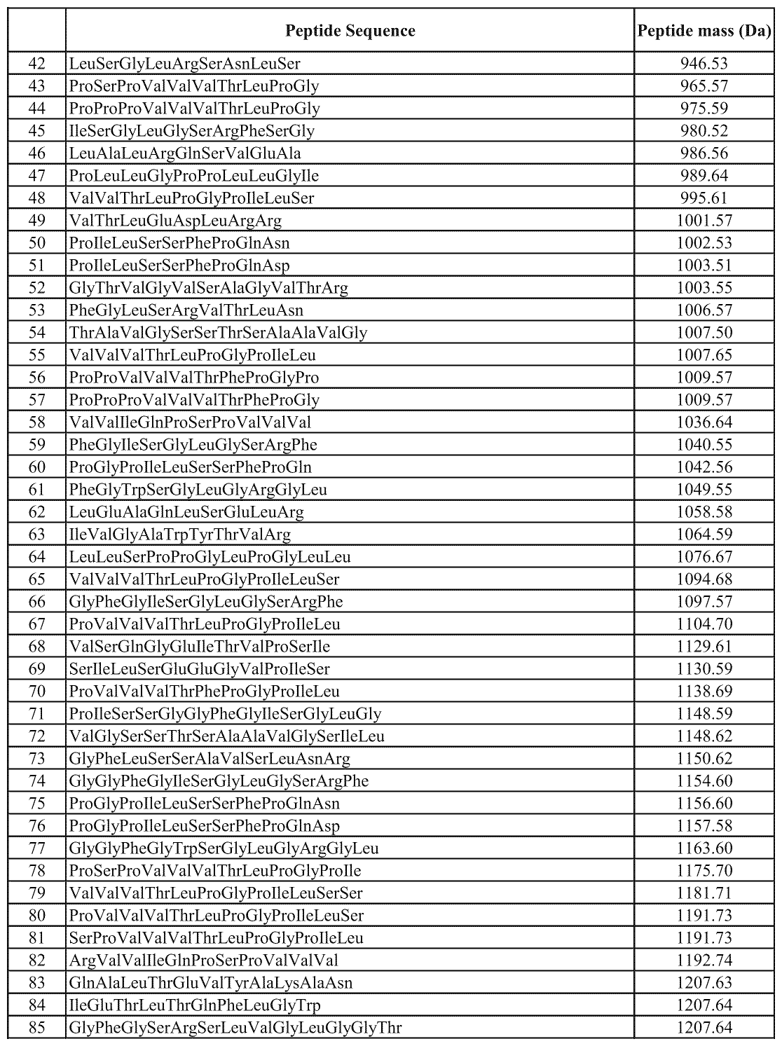

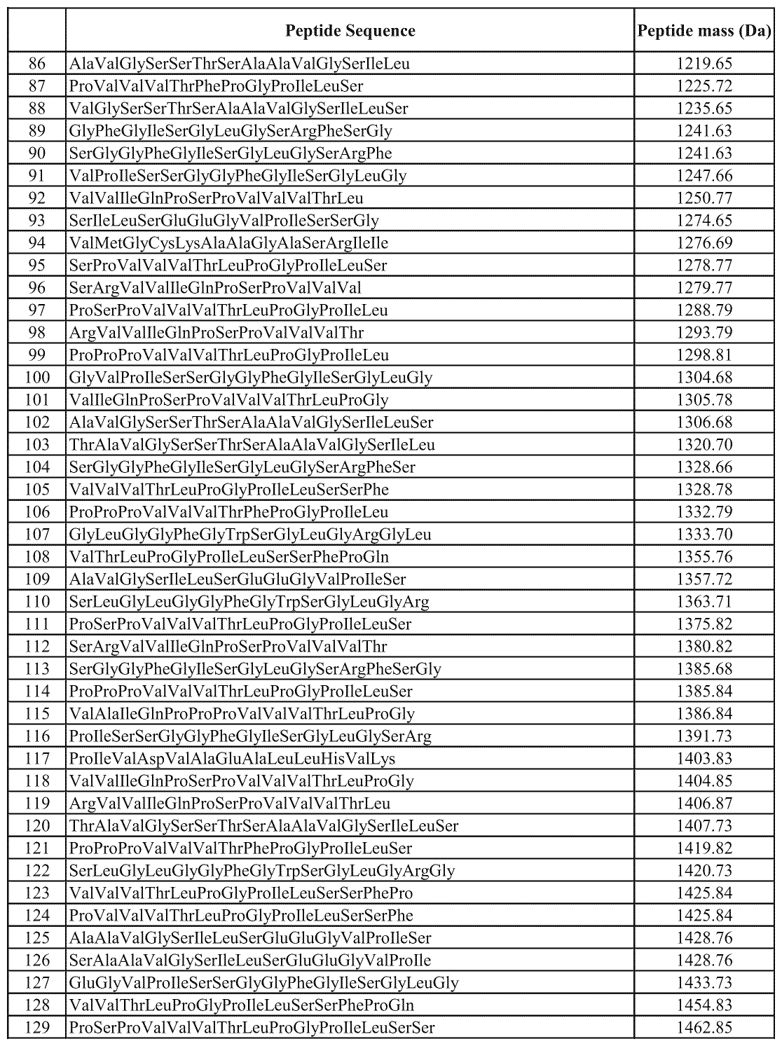

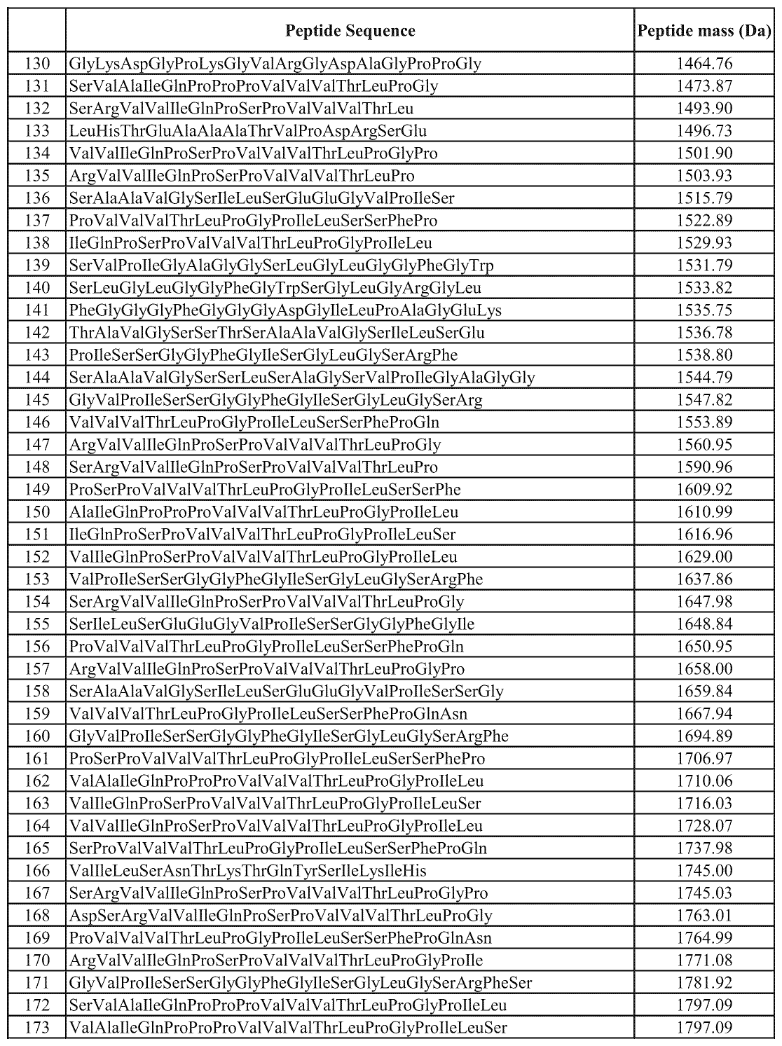

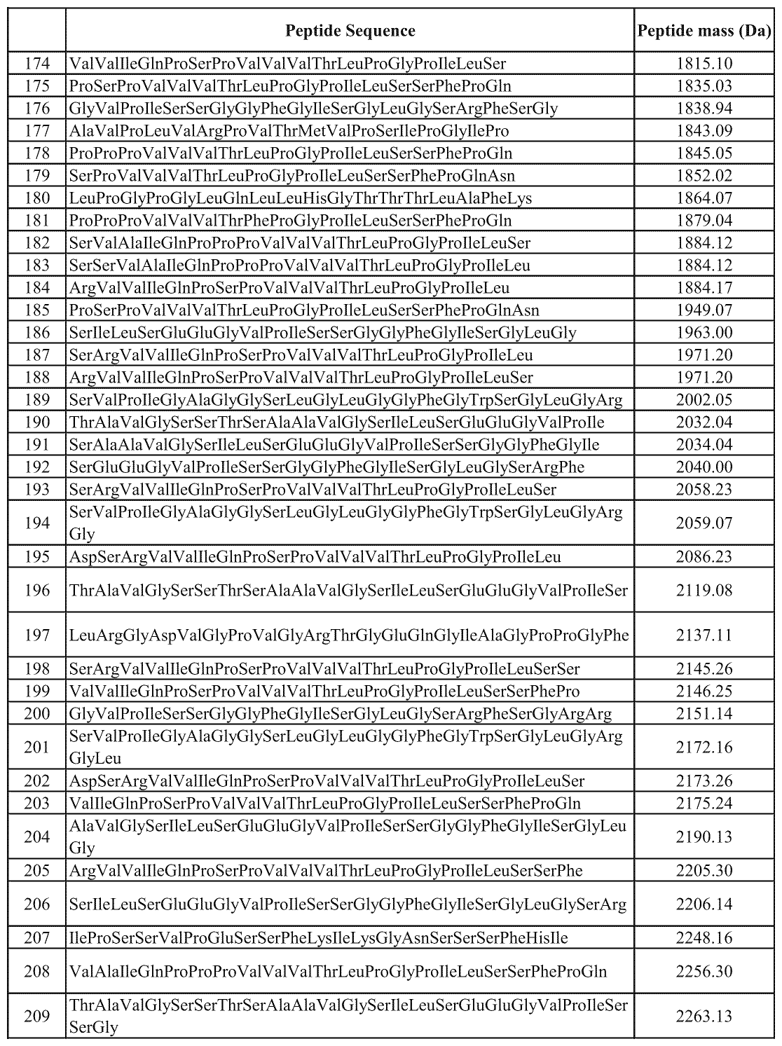

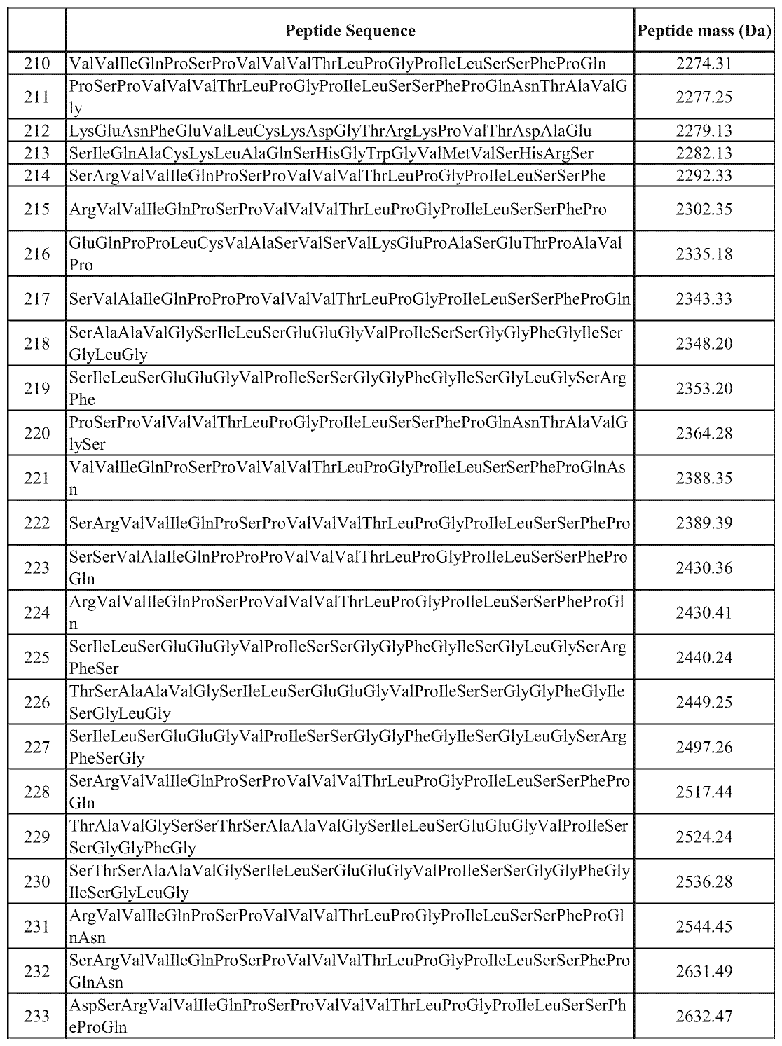

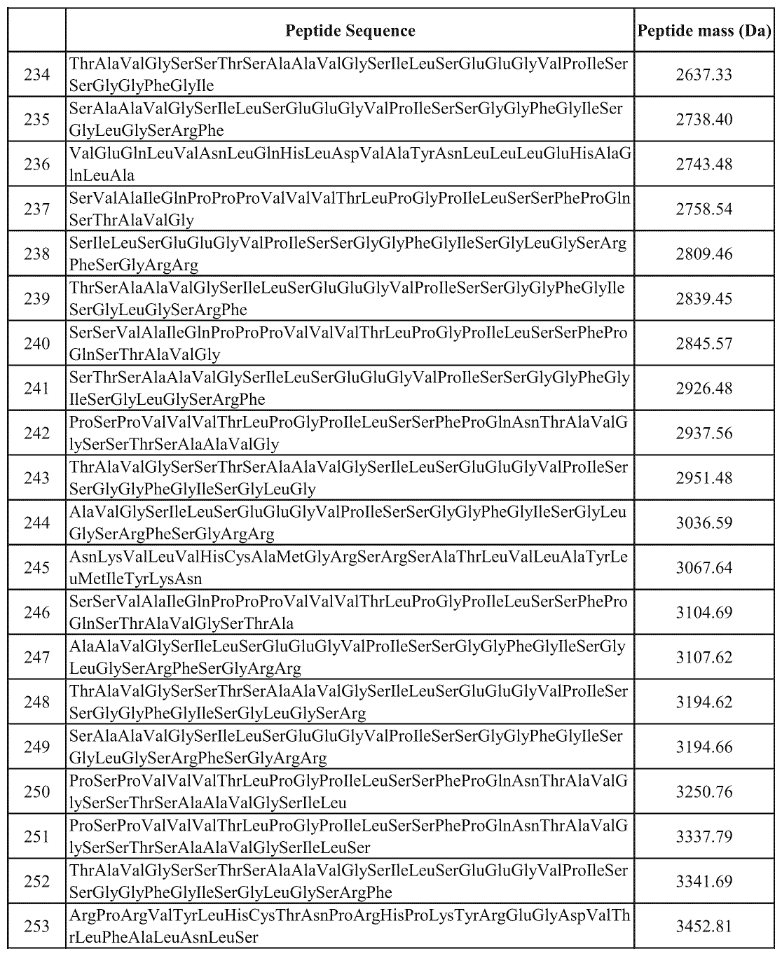

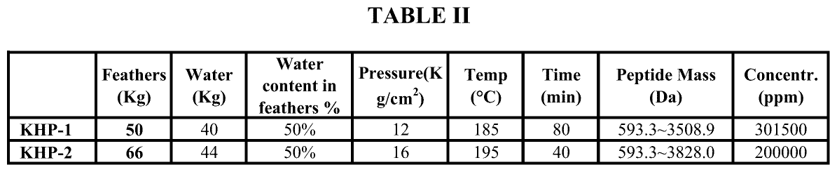

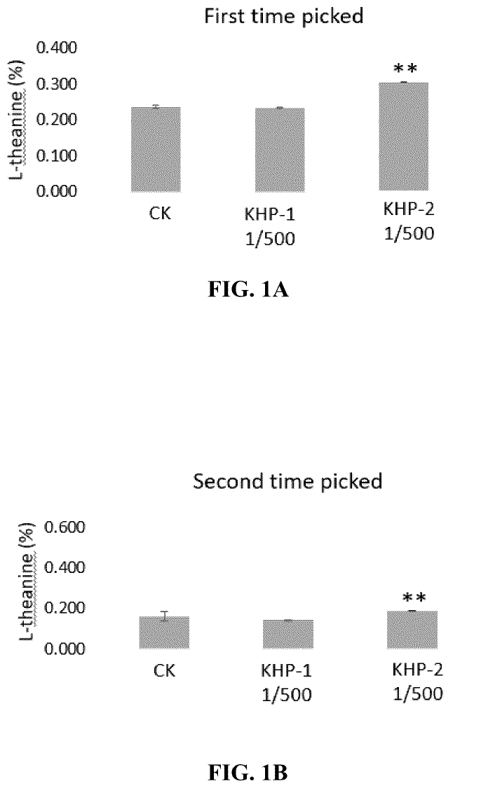

FIG. 1 shows the comparison of L-theanine content in the three groups. In the two pickings (FIG. 1A and FIG. 1B respectively), both KHP-2 group shows better L-theanine content than the CK group.

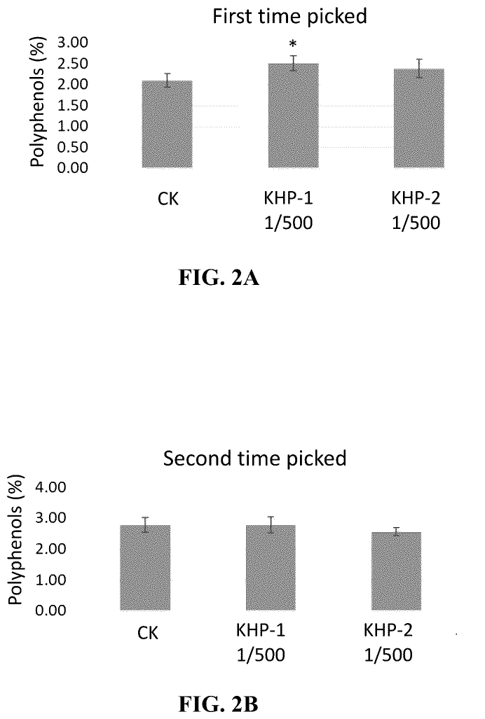

The harvested tea leaf samples are dipped into 70% methanol for 30 minutes, at a temperature of 70° C., after filtration and drying and diluted with ionic solution, the liquid is fed with 10% folin-ciocalteu phenol, and 7.5% sodium carbonate, waiting out 60 minutes for all reaction to end. Based upon standard curve of Gallic acid, the liquid is analyzed by an enzyme analyzer (Infinite M200 Pro, tecan) to test the light absorption of the 765 nm wave then to derive the polyphenol content shown in FIG. 2.

As shown in FIG. 2A and FIG. 2B, the polyphenol content is better in the KHP-1 group.

The inventors did more test on tea leaf samples from plants whose surrounding soil was infused with the KHP solutions are harvested, first time picking and second time picking similar to the way stated before. The infusion was done weekly, for a period of consecutive 4 weeks at the leaf sprouting stage. Four weeks after the fourth infusion, proceed with the harvesting (same first time and second time as stated before) to obtain tea leaf samples.

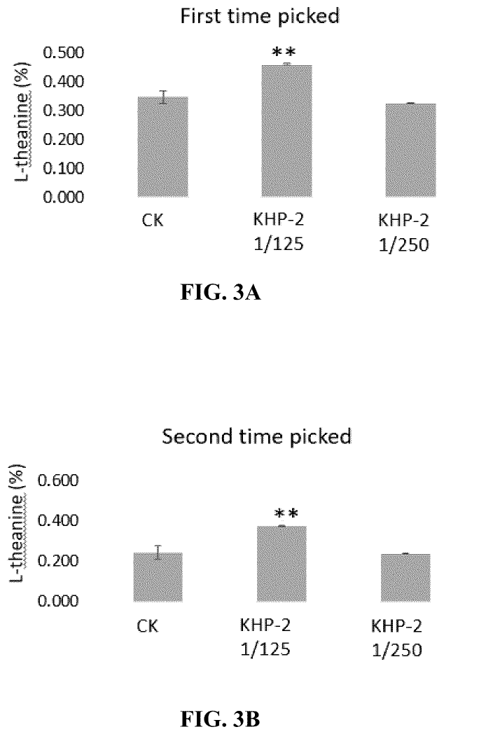

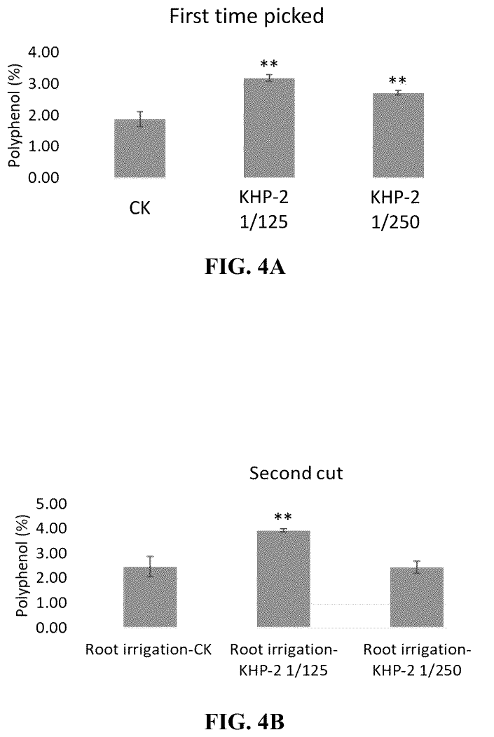

Using the same testing methodologies re L-theanine and polyphenol, the comparisons are shown in FIG. 3 and FIG. 4.

FIG. 3A and FIG. 3B show that KHP-2 solution, at dilution ratio of 1:125 produced higher L-theanine content.

FIG. 4A and FIG. 4B show that KHP-2 solution, at dilution ration of 1:125, produced higher polyphenol content.

As has been proven by the field tests, and the scientific analysis/measurement done by the inventors, the method of creating the KHP solution and the applications will help with the enhancement of the tea leaf's flavor.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. A method of using a keratin hydrolysis peptide (KHP) solution to a tea plant for the enhancement of the tea leaves' flavors, comprising the steps of:
a. Preparing the KHP solution by mixing 50 kg of feathers whose content is 50% water and 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to tea plants at leaf sprouting stage.

2. The method of using a keratin hydrolysis peptide (KHP) solution of claim 1 where the solution is diluted with water by volume at the ratio of 1:200-1,000 and is sprayed to the tea leaves' surface.

3. The method of using a keratin hydrolysis peptide (KHP) solution of claim 1 where the solution is diluted with water by volume at the ratio of 1:100-1,000 and is infused to the soil around the tea plants.

4. A method of using a keratin hydrolysis peptide (KHP) solution to a cotton plant for the enhancement of the plant's drought tolerance, comprising the steps of:
a. Preparing the KHP solution by mixing 66 kg of feathers whose content is 50% water and 44 kg of water in a sealed container;
b. hydrolyzing the mixture in the container with a temperature and pressure setting of 195° C. and 16 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to tea plants at leaf sprouting stage.

5. The method of using a keratin hydrolysis peptide (KHP) solution of claim 4 where the solution is diluted with water by volume at the ratio of 1:200-1,000 and is sprayed to the tea leaves' surface.

6. The method of using a keratin hydrolysis peptide (KHP) solution of claim 4 where the solution is diluted with water by volume at the ratio of 1:100-1,000 and is infused to the soil around the tea plants.
